(19) United States
(12) Patent Application Publication
(10) Pub. No.: US 2020/0277548 A1
(43) Pub. Date: Sep. 3, 2020

(54) Detergents And Cleaning Products Containing A Polymer Active Ingredient

(71) Applicant: Henkel AG & Co. KGaA, Duesseldorf (DE)

(72) Inventors: Benoit Luneau, Ratingen (DE); Regina Palkovits, Aachen (DE); Peter Hausoul, Landgraaf (NL); Carsten Stobbe, Aachen (DE)

(21) Appl. No.: 16/875,621

(22) Filed: May 15, 2020

Related U.S. Application Data
(63) Continuation of application No. PCT/EP2018/079974, filed on Nov. 2, 2018.

(30) Foreign Application Priority Data
Nov. 17, 2017 (DE) 102017010653.3

(51) Int. Cl. C11D 3/37; C08F 116/14; C11D 3/00; C11D 11/00; C07C 43/178

(52) U.S. Cl. CPC C11D 3/3753; C08F 116/14; C07C 43/178; C11D 11/0017; C11D 11/0023; C11D 3/0036

(57) ABSTRACT

A compound for improving the primary detergent power of detergents and cleaning products, in particular with respect to soiling containing oil and/or grease. This is achieved essentially by the incorporation of polymers obtainable by polymerization of compounds of the general formula (I), in which R¹, R² and R³, independently of one another, represent H or an alkyl group having 1 to 3 C atoms, with α,β-monoethylenically unsaturated carboxylic acids, carboxylic acid esters, carboxylic anhydrides, carboxamides, carboxylic acid imides, nitriles and their mixtures.

### FIELD OF THE INVENTION

The present invention relates to certain polymers obtainable from renewable raw materials, the monomers on which the renewable raw materials are based on, said monomers being accessible from renewable raw materials, the use of such polymers to enhance the primary detergent power of detergents or cleaning products when washing textiles or cleaning hard surfaces, as well as detergents and cleaning products containing such polymers.

### BACKGROUND OF THE INVENTION

In addition to the ingredients such as surfactants and builder materials that are essential to the washing process, detergents generally contain further constituents which can be subsumed under the term “active washing auxiliaries” and comprise the very different active ingredient groups such as foam regulators, graying inhibitors, bleaching products, bleach activators and dye transfer inhibitors. Auxiliaries of this kind also include substances, the presence of which enhances the detergent power of surfactants without generally needing to have a pronounced surfactant behavior themselves. The same applies mutatis mutandis to cleaning products for hard surfaces. Such substances are often referred to as detergent power enhancer.

International patent application WO 01/57171 A1 discloses detergents or rinsing products which, in addition to surfactant, comprise copolymers of anionic and cationic monomers and, if appropriate, additionally nonionic monomers.

Such copolymers have the disadvantage that they are essentially completely composed of monomers which are produced petrochemically. There is a need for performance enhancing polymers that are at least partially composed of monomers that can be made from renewable resources.

Japanese Patent JP 5288153 B discloses bisacetonides of 1,2,3,4,5-pentols whose hydroxyl group, which is not converted into the ketals, carries an allyl ether function.

Surprisingly, it has been found that polymers of 3-allyloxypentols and copolymers of 3-allyloxypentols with ethylenically unsaturated carboxylic acids have particularly good properties which enhance the performance of detergents and cleaning products.

### BRIEF SUMMARY OF THE INVENTION

A first subject of the invention is 3-allyloxy-1,2,4,5-tetrahydroxypentane and its derivatives of general formula (I)

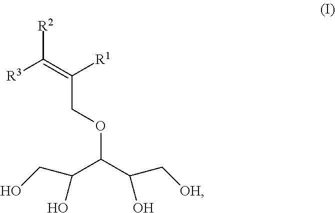

in which R¹, R² and R³ are independently H or an alkyl group having 1 to 3 carbon atoms, and their derivatives in which the hydroxyl groups are protected by conventional protecting groups, for example, as acetal such as tetrahydropyranyl ether, as ketal such as acetonide, or as carboxylic acid esters such as acetate.

Further objects of the invention are polymers obtainable by free-radical polymerization of compounds of the abovementioned general formula (I), and copolymers obtainable by free-radical copolymerization of compounds of the abovementioned general formula (I) with α,β-monoethylenically unsaturated carboxylic acids, carboxylic esters, carboxylic anhydrides, carboxamides, carboxylic imides, nitriles and mixtures thereof. In preferred compounds of the formula I, R¹, R² and R³ are the same; likewise, in preferred compounds of the formula I, at least one of the radicals R¹, R² and R³ is hydrogen.

Monomers of the general formula I are obtainable by reacting the middle hydroxyl group of xylitol or ribitol with allylation reagents, for example allyl bromide; for this, the remaining hydroxyl groups are expediently converted into an unreactive form and, for example, acid-catalyzed protected as acetonides, and released again the hydroxyl groups from these acetonides after allylation in position 1,2, 4 and 5.

They can be polymerized in the presence of conventional free-radical initiators such as azobisisobutyronitrile or benzoyl peroxide or copolymerized with ethylenically unsaturated carboxylic acids or carboxylic acid derivatives, wherein the two vicinal hydroxyl group pairs can be protected in the polymerization by conventional protecting groups, for example as acetal such as tetrahydropyran, as ketal such as acetonide or as a carboxylic acid ester such as acetate, and the protective groups are removed after the polymerization again.

The α,β-monoethylenically unsaturated carboxylic acids and their derivatives are preferably selected from acids such as acrylic acid, methacrylic acid, maleic acid, fumaric acid, esters such as dimethyl maleate, diethyl maleate, dimethyl fumarate, diethyl fumarate, methyl acrylate, ethyl acrylate, n-propyl acrylate, isopropyl acrylate, butyl acrylate, pentyl acrylate, hexyl acrylate, 2-ethylhexyl acrylate, nonyl acrylate, lauryl acrylate, trimethylcyclohexyl acrylate, t-butylcyclohexyl acrylate, benzyl acrylate, hydroxyethyl acrylate, ethoxyethyl acrylate, ethoxyethoxyethyl acrylate, aminoethyl acrylate, t-butylaminoethyl acrylate, N,N-dimethylaminoethyl acrylate, N,N-diethylaminoethyl acrylate, and corresponding methacrylates, amides such as maleic acid diamide, fumaric acid diamide, acrylamide, N-methylacrylamide, N-ethylacrylamide, Nn-propylacrylamide, N-i sopropylacrylamide, N-butylacrylamide, N-octylacrylamide, N-dodecylacrylamide, N-octadecylacrylamide, N-butoxymethylacrylamide, N,N-Dime ethylacrylamide, N,N-diethylacrylamide, N,N-dipropylacrylamide, N,N-dibutylacrylamide, N-(N′,N′-dimethylamino)ethylacrylamide, N-(N′,N′-diethylamino)ethylacrylamide, methacrylamide and corresponding N-substituted methacrylamides, anhydrides such as maleic anhydride, imides such as N-acroyl and N-methacroyl butyro, capro and valerolactam, maleimide, N-phenyl and N-methyl-maleimide, nitriles such as acetonitrile and fumarodinitrile, which may be used individually or as mixtures of two or more of these compounds.

Another object of the invention is the use of said polymers and/or copolymers to enhance the primary detergent power of detergents or cleaning products when washing textiles or when cleaning hard surfaces with respect to soiling. In particular in these, the copolymers according to the invention need not comprise carboxylic acid groups, carboxylic acid ester groups, carboxylic anhydride groups, carboxylic acid amide groups or carboxylic imide groups derived from the α,β-monoethylenically unsaturated monomers but these groups may be hydrolyzed in salt form, for example as sodium, potassium or ammonium carboxylate groups, in whole or in part, wherein the ammonium group may also be substituted by 1 to 4 alkyl or hydroxyalkyl groups or mixtures thereof.

The copolymers essential to the invention are accessible as described by free-radical copolymerization of the stated monomers, which can be carried out as a blockwise or preferably random copolymerization. They have no other units than units derived from the two monomers mentioned, with units derived from the radical initiator or from the radical termination reaction being able to be present at the polymer ends as a result of the preparation.

The polymer active ingredient according to the invention preferably has an average molecular weight (here and below in the case of average molecular weight data: number average) in the range from 1,000 g/mol to 100,000 g/mol, in particular from 1,500 g/mol to 50,000 g/mol. In the copolymer essential to the invention, the units derived from the compound of the general formula I and the units derived from the α,β-monoethylenically unsaturated carboxylic acid and/or derivatives thereof are preferably in molar ratios in the range from 4:1 to 1:4, in particular 2:1 to 1:2.

The use of the active ingredient according to the invention leads to a significantly better detachment of soiling on hard surfaces and on textiles, even those made of cotton or with a proportion of cotton, as is the case when using compounds known so far for this purpose. Alternatively, significant amounts of surfactants can be saved while retaining the ability to remove grease.

The use according to the invention can be carried out as part of a washing or cleaning process by adding the polymer essential to the invention to an aqueous liquor containing detergent or cleaning product or preferably incorporating it as a constituent of a detergent or cleaning product into the liquor, the concentration of the active ingredient in the liquor preferably being in the range from 0.005 g/l to 0.5 g/l, in particular from 0.02 g/l to 0.1 g/l.

Another object of the invention is a method for removing soiling from textiles or hard surfaces by contacting the garment or surface to be cleaned with an aqueous liquor containing a detergent or cleaning product and a said polymer active ingredient. This method can be carried out manually or mechanically, for example by means of a household washing machine or dishwasher. It is possible to use the particular liquid detergent or cleaning product and the active ingredient simultaneously or sequentially. The simultaneous application can be carried out particularly advantageously by the use of a product which contains the active ingredient.

A further subject of the invention is therefore a detergent or cleaning agent containing an above-defined polymer according to the invention.

### DETAILED DESCRIPTION OF THE INVENTION

Detergents or cleaning products which contain or are used together with an active substance to be used according to the invention or are used in the process according to the invention may contain all other customary constituents of such products which do not interact in an undesired manner with the active ingredient essential to the invention. Preferably, a polymer active ingredient as defined above is incorporated in detergents or cleaning products in amounts of from 0.1 wt.-% to 10 wt.-%, in particular 0.5 wt.-% to 2 wt.-%.

A product containing or used together with an active ingredient to be used or used in the method of the invention preferably contains synthetic anionic surfactant of sulfate and/or sulfonate type, especially alkylbenzenesulfonate, fatty alkyl sulfate, fatty alkyl ether sulfate, alkyl and/or dialkyl sulfosuccinate, sulfo fatty acid esters and/or sulfo fatty acid salts, in particular in an amount in the range of 2 wt.-% to 25 wt.-% and particularly preferably from 5 wt.-% to 15 wt.-%. The anionic surfactant is preferably selected from the alkylbenzenesulfonates, the alkyl or alkenyl sulfates and/or the alkyl or alkenyl ether sulfates in which the alkyl or alkenyl group has 8 to 22, in particular 12 to 18, carbon atoms. These are usually not individual substances, but cuts or mixtures. Of these, preference is given to those whose content of compounds having longer-chain radicals in the range from 16 to 18 carbon atoms is more than 20% by weight. Particular preference is given to the presence of the abovementioned combination of polymer essential to the invention and alkylbenzenesulfonate with linear C₉₋₁₃-alkyl groups in the products.

Another embodiment of such products comprises the presence of non-ionic surfactant selected from fatty alkyl polyglycosides, fatty alkyl polyalkoxylates, especially ethoxylates and/or propoxylates, fatty acid polyhydroxyamides and/or ethoxylation and/or propoxylation products of fatty alkylamines, vicinal diols, fatty acid alkyl esters and/or fatty acid amides and mixtures thereof, in particular in an amount in the range of 2 wt.-% to 25 wt.-%.

Suitable non-ionic surfactants include the alkoxylates, in particular the ethoxylates and/or propoxylates of saturated or mono- to polyunsaturated linear or branched-chain alcohols having 10 to 22 carbon atoms, preferably 12 to 18 carbon atoms. The degree of alkoxylation of the alcohols is generally between 1 and 20 and preferably between 3 and 10. They may be produced in known manner by reaction of the corresponding alcohols with the corresponding alkylene oxides. Particularly suitable are the derivatives of fatty alcohols, although their branched-chain isomers, in particular so-called oxo alcohols, can be used for the preparation of usable alkoxylates. Accordingly, the alkoxylates are useful, in particular the ethoxylates, primary alcohols with linear, in particular dodecyl, tetradecyl, hexadecyl or octadecyl radicals and mixtures thereof. In addition, suitable alkoxylation products of alkylamines, vicinal diols and carboxamides, which correspond to the said alcohols with respect to the alkyl part, are usable. In addition, the ethylene oxide and/or propylene oxide insertion products of fatty acid alkyl esters and fatty acid polyhydroxyamides can be considered. So-called alkylpolyglycosides which are suitable for incorporation in the compositions according to the invention are compounds of the general formula (G)ₙ—OR¹², in which is an alkyl or alkenyl radical having 8 to 22 carbon atoms, G is a glycose unit and n is a number between 1 and 10. The glycoside component (G)n are oligomers or polymers of naturally occurring aldose or ketose monomers, including in particular glucose, mannose, fructose, galactose, talose, gulose, altrose, allose, idose, ribose, arabinose, xylose and lyxose. The oligomers consisting of such glycosidically linked monomers are characterized not only by the nature of the sugars contained in them but also by their number, the so-called degree of oligomerization. The degree of oligomerization n assumes generally broken numerical values as the value to be analytically determined; it is in the range between 1 and 10, with the glycosides preferably used below a value of 1.5, in particular between 1.2 and 1.4. A preferred monomer building block is glucose because of its good availability. The alkyl or alkenyl moiety R¹² of the glycosides is preferably also derived from readily available derivatives of renewable raw materials, in particular from fatty alcohols, although their branched-chain isomers, in particular so-called oxo alcohols, can be used for the preparation of usable glycosides. Accordingly, the primary alcohols having linear octyl, decyl, dodecyl, tetradecyl, hexadecyl or octadecyl radicals and also mixtures thereof are particularly suitable. Particularly preferred alkyl glycosides contain a coconut oil alkyl radical, that is, mixtures having substantially R¹²=dodecyl and R¹²=tetradecyl.

Non-ionic surfactant is present in compositions which contain an active ingredient used according to the invention or are used in the scope of the use according to the invention, preferably in amounts of from 1% by weight to 30% by weight, in particular from 1% by weight to 25% by weight, with amounts in the upper part of this range being more likely to be found in liquid detergents and particulate detergents preferably containing lower amounts of up to 5% by weight.

The products may instead or additionally contain other surfactants, preferably synthetic anionic surfactants of the sulfate or sulfonate type. Suitable synthetic anionic surfactants which are particularly suitable for use in such compositions are, in addition to the abovementioned alkylbenzenesulfonates, the alkyl and/or alkenyl sulfates having 8 to 22 carbon atoms which carry an alkali metal, ammonium or alkyl or hydroxyalkyl-substituted ammonium ion as countercation. Preference is given to the derivatives of the fatty alcohols having in particular 12 to 18 carbon atoms and their branched-chain analogs, the so-called oxo alcohols. The alkyl and alkenyl sulfates can be prepared in a known manner by reaction of the corresponding alcohol component with a conventional sulfating reagent, in particular sulfur trioxide or chlorosulfonic acid, and subsequent neutralization with alkali, ammonium or alkyl or hydroxyalkyl-substituted ammonium bases. Sulfur-type surfactants which can be used also include the sulfated alkoxylation products of the alcohols mentioned, known as ether sulfates. Such ether sulfates preferably contain from 2 to 30, in particular from 4 to 10, ethylene glycol groups per molecule. Suitable anionic surfactants of the sulfonate type include the α-sulfoesters obtainable by reaction of fatty acid esters with sulfur trioxide and subsequent neutralization, in particular those of fatty acids having 8 to 22 carbon atoms, preferably 12 to 18 carbon atoms, and linear alcohols of 1 to 6 carbon atoms, preferably 1 to 4 carbon atoms, derivative sulfonation products, as well as the formal saponification resulting from these sulfo fatty acids. Preferred anionic surfactants are also the salts of sulfosuccinic acid esters, which are also referred to as alkylsulfosuccinates or dialkylsulfosuccinates, and which are monoesters or diesters of sulfosuccinic acid with alcohols, preferably fatty alcohols and in particular ethoxylated fatty alcohols. Preferred sulfosuccinates contain C₈ to C₁₈ fatty alcohol residues or mixtures of these. Particularly preferred sulfosuccinates contain an ethoxylated fatty alcohol radical, which in itself is a nonionic surfactant. Among these, in turn, sulfosuccinates including fatty alcohol groups that derive from ethoxylated fatty alcohols exhibiting a restricted distribution of homologs are particularly preferred.

Other optional surface-active ingredients include soaps, in which saturated fatty acid soaps, such as the salts of lauric acid, myristic acid, palmitic acid or stearic acid, as well as soaps derived from natural fatty acid mixtures, for example coconut, palm kernel or tallow fatty acids, are suitable. In particular, those soap mixtures are preferred which are composed of 50% to 100% by weight of saturated C₁₂-C₁₈ fatty acid soaps and up to 50% by weight of oleic acid soap. Preferably, soap is included in amounts of from 0.1% to 5% by weight. In particular, in liquid products containing an active ingredient according to the invention, however, higher amounts of soap can be contained, usually up to 20 wt.-%.

If desired, the compositions may also contain betaine surfactants and/or cationic surfactants which, if present, are preferably used in amounts of from 0.5% to 7% by weight. Among them, the esterquats discussed below are particularly preferred.

If desired, the compositions may contain peroxygen based bleaching products, in particular in amounts ranging from 5% to 70% by weight, and optionally bleach activators, especially in amounts ranging from 2% to 10% by weight. The bleaching products in question are preferably the peroxygen compounds generally used in detergents, such as percarboxylic acids, for example dodecanedioic acid or phthaloylaminoperoxicaproic acid, hydrogen peroxide, alkali metal perborate, which may be in the form of tetra- or monohydrate, percarbonate, perpyrophosphate and persilicate, which are generally used as alkali metal salts, in particular as sodium salts. Such bleaching products are present in detergents containing an active ingredient used according to the invention, preferably in amounts of up to 25 wt.-%, in particular up to 15% by weight and particularly preferably from 5 wt.-% to 15 wt.-%, respectively on total product, wherein in particular percarbonate is used. The optionally present component of the bleach activators comprises the commonly used N- or O-acyl compounds, for example polyacylated alkylenediamines, in particular tetraacetylethylenediamine, acylated glycolurils, in particular tetraacetylglycoluril, N-acylated hydantoins, hydrazides, triazoles, urazoles, diketopiperazines, sulphurylamides and cyanurates, also carboxylic acid anhydrides, in particular phthalic acid anhydride, carboxylic acid esters, in particular sodium isononanoyl-phe-nolsulfonat, and acylated sugar derivatives, in particular pentaacetylglucose, and cationic nitrile derivatives such as trimethylammoniumacetonitrile salts. The bleach activators may have been coated and/or granulated in a known manner with coating substances in order to avoid the interaction with the per compounds, wherein granulated 1,5-diacetyl-2,4-dioxohexahydro-1,3,5-triazine, and/or trialkylammonium acetonitrile in particulate form, produced with the aid of granulated tetraacetylethylenediamine having mean particle sizes of from 0.01 mm to 0.8 mm, is particularly preferred. Such bleach activators are preferably contained in detergents in amounts of up to 8% by weight, in particular from 2% by weight to 6% by weight, based in each case on the total product.

In a further embodiment, the composition contains water-soluble and/or water-insoluble builder, in particular selected from alkali metal aluminosilicate, crystalline alkali metal silicate with a module above 1, monomeric polycarboxylate, polymeric polycarboxylate and mixtures thereof, in particular in amounts ranging from 2.5 wt.-% to 60 wt.-%.

The product preferably contains from 20% to 55% by weight of water-soluble and/or water-insoluble, organic and/or inorganic builders. The water-soluble organic builder substances include, in particular, those from the class of the polycarboxylic acids, in particular citric acid and sugar acids, and the polymeric (poly) carboxylic acids, in particular the polycarboxylates obtainable by oxidation of polysaccharides, polymeric acrylic acids, methacrylic acids, maleic acids and mixed polymers thereof, which may also contain polymerized small amounts of polymerizable substances without carboxylic acid functionality. The relative molecular mass of the homopolymers of unsaturated carboxylic acids is generally between 5,000 g/mol and 200,000 g/mol, that of the copolymers between 2,000 g/mol and 200,000 g/mol, preferably 50,000 g/mol to 120,000 g/mol, based on free acid. A particularly preferred acrylic acid-maleic acid copolymer has a relative molecular mass of from 50,000 to 100,000. Compounds of this class which are suitable, although less preferred, are copolymers of acrylic acid or methacrylic acid with vinyl ethers, such as vinyl methyl ethers, vinyl esters, ethylene, propylene, and styrene, in which the proportion of the acid is at least 50 wt. %. It is also possible to use, as water-soluble organic builder substances, terpolymers which contain two carboxylic acids and/or the salts thereof as monomers and vinyl alcohol and/or a vinyl alcohol derivative or a carbohydrate as the third monomer. The first acid monomer or the salt thereof is derived from a monoethylenically unsaturated C₃-C₈ carboxylic acid and preferably from a C₃-C₄ monocarboxylic acid, in particular from (meth)acrylic acid. The second acid monomer or the salt thereof can be a derivative of a C₄-C₈ dicarboxylic acid, maleic acid being particularly preferred. The third monomeric unit is formed in this case of vinyl alcohol and/or preferably an esterified vinyl alcohol. In particular, vinyl alcohol derivatives are preferred which are an ester of short-chain carboxylic acids, for example C₁-C₄ carboxylic acids, with vinyl alcohol. Preferred terpolymers contain 60 wt. % to 95 wt. %, in particular 70 wt. % to 90 wt. %, (meth)acrylic acid and/or (meth)acrylate, particularly preferably acrylic acid and/or acrylate, and maleic acid and/or maleate, and 5 wt. % to 40 wt. %, preferably 10 wt. % to 30 wt. %, vinyl alcohol and/or vinyl acetate. Very particularly preferred are terpolymers in which the weight ratio of (meth)acrylic acid and/or (meth)acrylate to maleic acid and/or maleate is between 1:1 and 4:1, preferably between 2:1 and 3:1, and in particular between 2:1 and 2.5:1. Both the amounts and the weight ratios are based on the acids. The second acid monomer or the salt thereof can also be a derivative of an allylsulfonic acid which is substituted in the 2 position with an alkyl radical, preferably with a C₁-C₄ alkyl radical, or an aromatic radical which is preferably derived from benzene or benzene derivatives. Preferred terpolymers contain from 40 wt. % to 60 wt. %, in particular from 45 to 55 wt. %, (meth)acrylic acid and/or (meth)acrylate, particularly preferably acrylic acid and/or acrylate, from 10 wt. % to 30 wt. %, preferably 15 wt. % to 25 wt. %, methallylsulfonic acid and/or methallylsulfonate and 15 wt. % to 40 wt. %, preferably 20 wt. % to 40 wt. %, of a carbohydrate as a third monomer. This carbohydrate may be, for example, a mono-, di-, oligo- or polysaccharide, mono-, di- or oligosaccharides being preferred, sucrose particularly being preferred. The use of the third monomer presumably incorporates predetermined breaking points into the polymer which are responsible for the good biodegradability of the polymer. These terpolymers generally have a molecular weight between 1000 g/mol and 200,000 g/mol, preferably between 2000 g/mol and 50,000 g/mol and in particular between 3000 g/mol and 10,000 g/mol. The organic builder substances may, in particular for the preparation of liquid products, be used in the form of aqueous solutions, preferably in the form of 30 to 50 wt. % aqueous solutions. All mentioned polycarboxylic acid are generally used in the form of the water-soluble salts thereof, in particular the alkali salts thereof.

Organic builder substances of this kind can, preferably, be contained in amounts of up to 40 wt. %, in particular up to 25 wt. %, and particularly preferably from 1 wt. % to 5 wt. %. Amounts close to the stated upper limit are preferably used in paste-form or liquid, in particular water-containing, products.

In particular crystalline or amorphous alkali aluminosilicates are used as water-insoluble, water-dispersible inorganic builder materials in amounts of up to 50 wt. %, preferably no greater than 40 wt. %, and in liquid products in particular in amounts of from 1 wt. % to 5 wt. %. Among these, the detergent-grade crystalline aluminosilicates, especially zeolite NaA and optionally NaX, are preferred. Amounts close to the stated upper limit are preferably used in solid particulate products. Suitable aluminosilicates have in particular no particles having a particle size greater than 30 μm and preferably consist up to at least 80 wt. % of particles having a size smaller than 10 μm. The calcium binding capacity of said aluminosilicates is generally in the range of from 100 to 200 mg CaO per gram. Suitable substitutes or partial substitutes for the stated aluminosilicate are crystalline alkali silicates, which may be present alone or in a mixture with amorphous silicates. The alkali silicates that can be used in the products as builders preferably have a molar ratio of alkali oxide to SiO₂ of less than 0.95, in particular from 1:1.1 to 1:12, and may be present in amorphous or crystalline form. Preferred alkali silicates are sodium silicates, in particular amorphous sodium silicates, having a Na₂O:SiO₂ molar ratio of from 1:2 to 1:2.8. Such amorphous alkali silicates are commercially available, for example, under the name Porta®.

Those with a molar ratio of Na₂O: SiO₂ of 1:1.9 to 1:2.8 are preferably added in the course of the production as a solid and not in the form of a solution. Crystalline phyllosilicates of general formula Na₂SiₓO₂ₓ₊₁ yH₂O, where x, referred to as the module, is a number from 1.9 to 4, y is a number from 0 to 20, and preferred values for x are 2, 3 or 4, are preferably used as crystalline silicates, which may be present alone or in a mixture with amorphous silicates. Crystalline layered silicates which fall under this general formula are described, for example, in European Patent Application EP 0 164 514. Preferred crystalline phyllosilicates are those in which x in the stated general formula assumes the values 2 or 3. In particular, both β- and δ-sodium disilicates (Na₂Si₂O_{5·y}H₂O) are preferred. Practically water-free crystalline alkali silicates which have the above general formula, in which x is a number from 1.9 to 2.1, and which are prepared from amorphous alkali silicates may also be used in products which contain an active substance to be used according to the invention. In a further preferred embodiment of products according to the invention, a crystalline sodium phyllosilicate having a module of from 2 to 3, as can be produced from sand and soda, is used. Crystalline sodium silicates having a module in the range of from 1.9 to 3.5 are used in a further preferred embodiment of detergents according to the invention. The content of alkali metal silicates is preferably 1% by weight to 50% by weight and in particular 5% by weight to 35% by weight, based on anhydrous active substance. If alkali metal aluminosilicate, in particular zeolite, is present as an additional builder substance, the content of alkali silicate is preferably 1% by weight to 15% by weight and in particular 2% by weight to 8% by weight, based on anhydrous active substance. The weight ratio of aluminosilicate to silicate, in each case based on anhydrous active substances, is then preferably 4:1 to 10:1. In products containing both amorphous and crystalline alkali silicates, the weight ratio of amorphous alkali silicate to crystalline alkali silicate is preferably from 1:2 to 2:1 and in particular from 1:1 to 2:1.

In addition to the said inorganic builder, other water-soluble or water-insoluble inorganic substances may be contained in the products containing an active ingredient to be used in the present invention, used together with the same or used in the method according to the invention. Suitable in this context are the alkali metal carbonates, alkali metal bicarbonates and alkali metal sulfates and mixtures thereof. Such additional inorganic material may be present in amounts up to 70% by weight.

In addition, the products may contain other ingredients customary in detergents or cleaning products. These optional ingredients include, in particular, enzymes, enzyme stabilizers, complexing products for heavy metals, for example aminopolycarboxylic acids, aminohydroxypolycarboxylic acids, polyphosphonic acids and/or aminopolyphosphonic acids, foam inhibitors, for example organopolysiloxanes or paraffins, solvents and optical brighteners, for example stilbene disulfonic acid derivatives. Preferably, products which contain an active ingredient used according to the invention, contain up to 1% by weight, in particular 0.01% by weight to 0.5% by weight, of optical brighteners, in particular compounds from the class of the substituted 4,4′-Bis(2,4,6-tri-amino-s-triazinyl)-stilbene-2,2′-disulphonic acids, up to 5% by weight, in particular from 0.1% by weight to 2% by weight of complexing agent for heavy metals, in particular aminoalkylenephosphonic acids and salts thereof and up to 2% by weight, in particular from 0.1% by weight to 1% by weight, of foam inhibitors, the weight proportions in each case referring to the total product.

Solvents which can be used in particular for liquid products are, in addition to water, preferably those which are water-miscible. These include the lower alcohols, for example, ethanol, propanol, isopropanol, and the isomeric butanols, glycerol, lower glycols, such as ethylene and propylene glycol, and the ethers derivable from said classes of compounds. In such liquid products, the active compounds used in the invention are usually dissolved or in suspended form.

Optionally present enzymes are preferably selected from the group comprising protease, amylase, lipase, cellulase, hemicellulase, oxidase, peroxidase, pectinase and mixtures thereof. First and foremost, proteases derived from microorganisms, such as bacteria or fungi, come into question. It can be obtained in a known manner by fermentation processes from suitable microorganisms. Proteases are commercially available, for example, under the names BLAP®, Savinase®, Esperase®, Maxatase®, Optimase®, Alcalase®, Durazym® or Maxapem®. The lipase which can be used can be obtained, for example, from Humicola lanuginosa, from Bacillus species, from Pseudomonas species, from Fusarium species, from Rhizopus species or from Aspergillus species. Suitable lipases are commercially available, for example, under the names Lipolase®, Lipozym®, Lipomax®, Lipex®, Amano®-Lipase, Toyo-Jozo®-Lipase, Meito®-Lipase and Diosynth®-Lipase. Suitable amylases are commercially available, for example, under the names Maxamyl®, Termamyl®, Duramyl® and Purafect® OxAm. The usable cellulase may be an enzyme recoverable from bacteria or fungi, which has a pH optimum, preferably in the weakly acidic to slightly alkaline range of 6 to 9.5. Such cellulases are commercially available under the names Celluzyme®, Carezyme® and Ecostone®. Suitable pectinases that are suitable in this regard are available for example under the names Gamanase®, Pektinex AR®, XPect® or Pectaway® from Novozymes, under the names Rohapect UF®, Rohapect TPL®, Rohapect PTE100®, Rohapect MPE®, Rohapect MA plus HC, Rohapect DA12L®, Rohapect 10L®, Rohapect B1L® from AB Enzymes, and under the name Pyrolase® from Diversa Corp., San Diego, Calif., USA.

The customary enzyme stabilizers, present if appropriate, in particular in liquid products, include amino alcohols, for example mono-, di-, triethanol- and -propanolamine and mixtures thereof, lower carboxylic acids, boric acid, alkali borates, boric acid-carboxylic acid combinations, boric acid esters, boronic acid derivatives, calcium salts, for example, ca-formic acid combination, magnesium salts, and/or sulfur-containing reducing agents.

Suitable foam inhibitors include long-chain soaps, especially behenic soap, fatty acid amides, paraffins, waxes, microcrystalline waxes, organopolysiloxanes and mixtures thereof, which moreover can contain microfine, optionally silanated or otherwise hydrophobicized silica. For use in particulate products, such foam inhibitors are preferably bound to granular, water-soluble carrier substances.

The known polyester-active soil release polymers which can be used in addition to the active ingredients of the invention include copolyesters of dicarboxylic acids, for example adipic acid, phthalic acid or terephthalic acid, diols, for example ethylene glycol or propylene glycol, and polydiols, for example polyethylene glycol or polypropylene glycol. Preferred soil release polyesters include those compounds which are formally accessible by esterification of two monomeric moieties, wherein the first monomer is a dicarboxylic acid HOOC—Ph—COOH and the second monomer is a diol HO—(CHR¹¹—)ₐOH, which can also be present as polymeric diol H—(O—(CHR¹¹—)ₐ)_{b}OH. Therein, Ph is an o-, m- or p-phenylene radical which can carry from 1 to 4 substituents selected from alkyl functional groups having from 1 to 22 carbon atoms, sulfonic acid groups, carboxyl groups, and mixtures thereof, R¹¹ is hydrogen, an alkyl radical having from 1 to 22 carbon atoms and mixtures thereof, a is a number from 2 to 6 and b is a number from 1 to 300. Preferably, in the polyesters obtainable from these, both monomer diol units —O—(CHR¹¹—)ₐO— and also polymeric diol units —(O—(CHR¹¹—)ₐ)_{b}O— are present. The molar ratio of monomer diol units to polymer diol units is preferably 100:1 to 1:100, in particular 10:1 to 1:10. In the polymer diol units, the degree of polymerization b is preferably in the range from 4 to 200, in particular from 12 to 140. The molecular weight or the average molecular weight or the maximum of the molecular weight distribution of preferred soil release polyesters is in the range from 250 g/mol to 100,000 g/mol, in particular from 500 g/mol to 50,000 g/mol. The acid underlying the radical Ph is preferably selected from terephthalic acid, isophthalic acid, phthalic acid, trimellitic acid, mellitic acid, the isomers of sulfophthalic acid, sulfoisophthalic acid and sulfoterephthalic acid and mixtures thereof. If their acid groups are not part of the ester bonds in the polymer, they are preferably in salt form, in particular as alkali or ammonium salt. Among these, the sodium and potassium salts are particularly preferable. If desired, instead of the monomer HOOC—Ph—COOH, small proportions, in particular not more than 10 mol-% based on the proportion of Ph having the meaning given above, of other acids having at least two carboxyl groups may be present in the soil-release capable polyester. These include, for example, alkylene and alkenylene dicarboxylic acids such as malonic acid, succinic acid, fumaric acid, maleic acid, glutaric acid, adipic acid, pimelic acid, suberic acid, azelaic acid and sebacic acid. The preferred diols HO—(CHR¹¹—)ₐOH include those in which R¹¹.

is hydrogen and a is a number from 2 to 6, and those in which a has the value 2 and R¹¹ is selected from hydrogen and the alkyl radicals having 1 to 10, in particular 1 to 3, carbon atoms. Among the latter diols, those of the formula HO—CH₂—CHR¹¹—OH in which R¹¹ has the abovementioned meaning are particularly preferred. Examples of diol components are ethylene glycol, 1,2-propylene glycol, 1,3-propylene glycol, 1,4-butanediol, 1,5-pentanediol, 1,6-hexanediol, 1,8-octanediol, 1,2-decanediol, 1,2-dodecanediol and neopentyl glycol. Particularly preferred among the polymeric diols is polyethylene glycol having an average molecular weight in the range from 1000 g/mol to 6000 g/mol.

If desired, these polyesters composed as described above may also be end-capped, wherein alkyl groups having 1 to 22 carbon atoms and esters of monocarboxylic acids are suitable as end groups. The ester groups bonded via end groups can be based on alkyl, alkenyl and aryl monocarboxylic acids having 5 to 32 carbon atoms, in particular 5 to 18 carbon atoms. These include valeric acid, caproic acid, enanthic acid, caprylic acid, pelargonic acid, capric acid, undecanoic acid, undecenoic acid, lauric acid, lauroleinic acid, tridecanoic acid, myristic acid, myristoleic acid, pentadecanoic acid, palmitic acid, stearic acid, petroselic acid, petroselaidic acid, oleic acid, linoleic acid, linolaidic acid, linolenic acid, eleostearic acid, arachidic acid, gadoleic acid, arachidonic acid, behenic acid, erucic acid, brassic acid, clupanodonic acid, lignoceric acid, cerotic acid, melissic acid, benzoic acid, which may carry 1 to 5 substituents having a total of up to 25 carbon atoms, in particular 1 to 12 carbon atoms, for example tert-butylbenzoic acid. The end groups can also be based on hydroxymonocarboxylic acids having 5 to 22 carbon atoms, which include, for example, hydroxyvaleric acid, hydroxycaproic acid, ricinoleic acid, the hydrogenation product of which includes hydroxystearic acid and o-, m- and p-hydroxybenzoic acid. The hydroxymonocarboxylic acids may in turn be linked to one another via their hydroxyl group and their carboxyl group and thus be present several times in an end group. Preferably, the number of hydroxymonocarboxylic acid units per end group, that is to say their degree of oligomerization, is in the range from 1 to 50, in particular from 1 to 10. In a preferred embodiment of the invention, polymers of ethylene terephthalate and polyethylene terephthalate, in which the polyethylene glycol units have molecular weights of 750 g/mol to 5000 g/mol and the molar ratio of ethylene terephthalate to polyethylene oxide terephthalate is 50:50 to 90:10, are used in combination with an active ingredient substantial to the invention.

The soil release polymers are preferably water-soluble, the term “water-soluble” being understood to mean a solubility of at least 0.01 g, preferably at least 0.1 g, of the polymer per liter of water at room temperature and pH 8. However, preferably used polymers have a solubility of at least 1 g per liter, in particular at least 10 g per liter under these conditions.

The preparation of solid products according to the invention presents no difficulties and can be carried out in a known manner, for example by spray-drying or granulation, enzymes and possibly other thermally sensitive ingredients such as, for example, bleaching products optionally being added separately later. For the preparation of products according to the invention having an increased bulk weight, in particular in the range of from 650 g/l to 950 g/l, a method having an extrusion step is preferred.

For the preparation of compositions according to the invention in tablet form, which may be monophasic or multiphasic, monochromatic or multicolor and in particular may consist of one or more layers, in particular two layers, the procedure is preferably such that all constituents are mixed together—if appropriate one per layer—in one mixer and the mixture is pressed by means of conventional tablet presses, such as eccentric or rotary presses, with compressive forces in the range of about 50 to 100 kN, preferably at 60 to 70 kN. Particularly in the case of multilayer tablets, it may be advantageous if at least one layer is pre-compressed. This is preferably carried out at pressing forces between 5 and 20 kN, in particular at 10 to 15 kN. This gives fracture-resistant, yet sufficiently rapidly soluble tablets under application conditions with fracture and flexural strengths of normally 100 to 200 N, but preferably above 150 N. Preferably, a tablet produced in this way has a weight of 10 g to 50 g, in particular 15 g up to 40 g. The spatial form of the tablets is arbitrary and can be round, oval or angular, with intermediate forms also being possible. Corners and edges are advantageously rounded. Round tablets preferably have a diameter of 30 mm to 40 mm. In particular, the size of rectangular or cuboid-shaped tablets, which are predominantly introduced via the metering device, for example the dishwasher, is dependent on the geometry and the volume of this metering device. Exemplary preferred embodiments have a base area of (20 to 30 mm)×(34 to 40 mm), in particular of 26×36 mm or 24×38 mm.

Liquid or pasty products according to the invention containing solutions in the form of typical solvents, in particular water, are usually prepared by simple mixing of the ingredients, which can be put into an automatic mixer in substance or as a solution.

In a preferred embodiment, a product which is incorporated into the active ingredient to be used according to the invention is liquid and contains 1% by weight to 15% by weight, in particular 2% by weight to 10% by weight, of non-ionic surfactant, 2% by weight to 30% by weight, in particular 5% by weight to 20% by weight of synthetic anionic surfactant, up to 15% by weight, in particular 2% by weight to 12.5% by weight of soap, 0.5 wt.-% to 5 wt.-%, in particular 1 wt.-% to 4 wt.-% organic builder, in particular polycarboxylate such as citrate, up to 1.5 wt.-%, in particular 0.1 wt.-% to 1% by weight of complexing product for heavy metals, such as phosphonate, and optionally enzyme, enzyme stabilizer, color and/or perfume, water and/or water-miscible solvent.

In a further preferred embodiment, a product in which the active ingredient to be used according to the invention is incorporated is particulate and contains up to 25% by weight, in particular from 5% by weight to 20% by weight, of bleaching product, in particular alkali percarbonate, up to 15% by weight.-%, in particular 1 wt.-% to 10 wt.-% of bleach activator, 20 wt.-% to 55 wt.-% of inorganic builder, up to 10 wt.-%, in particular 2 wt.-% to 8 wt.-% of water-soluble organic builder, 10 wt.-% to 25 wt.-% of synthetic anionic surfactant, 1 wt.-% to 5 wt.-% of non-ionic surfactant and up to 25 wt.-%, in particular 0.1 Wt.-% to 25 wt.-% of inorganic salts, in particular alkali carbonate and/or alkali bicarbonate.

### EXAMPLES

### Example 1

### Monomer Production

a) Preparation of (4-(2,2-dimethyl-1,3-dioxolan-4-yl)hydroxymethyl)-2,2-dimethyl-1,3-dioxolane

A mixture of 50.00 g of xylitol (328.6 mmol) with 350 mL of acetone (4.76 mol, 5 15 eq) and 100 μL of 98 percent by weight aqueous H2SO4 was heated to 90° C. for 72 h. Then 2 spatulas of NaHCO₃ were added and the excess was filtered off on this neutralization agent. The neutralized reaction mixture was concentrated on a rotary evaporator until a light-yellow syrup remained. This was distilled at 150° C. in a high vacuum (2.0×10⁻³ mbar). 59.8 g of product were obtained as a colorless syrup with a yield of 79%.

δ[¹H, CDCl₃]=1.30 (d, 3H); 1.36 (d, 6H); 3.49-3.62 (m, 1H), 3.68-3.85 (m, 2H); 3.85-4.03 (m, 2H); 4.11 (dtd, 1H) ppm

δ[¹³C, CDCl₃]=25.16; 25.40; 26.09; 26.32; 26.90; 27.06; 62.11; 65.53; 65.86; 71.53; 75.05; 75.97; 76.89; 77.72; 109.42; 109.53; 109.66 ppm

b) Preparation of 4-(allyloxy-(2,2-dimethyl-1,3-dioxolan-4-yOmethyl)-2,2-dimethyl-1,3-dioxolane

A solution of 15.1 g (64.9 mmol) of the bisacetonide obtained in example 1a) in 40 ml of dimethylformamide (DMF) was added portionwise with 1.40 g of sodium hydride (58.4 mmol, 0.9 eq.) and stirred for about 5 min until no more bubbling was visible. Then, the reaction mixture was cooled to 0° C., added dropwise with 7.3 ml of allyl bromide (84.4 mmol, 1.3 eq.) and stirred at room temperature for 48 h. Subsequently, the reaction mixture was extracted with 30 ml deionized water and 2×30 ml diethyl ether. The combined organic phases were washed with 20 ml of saturated NaHCO₃ and saturated NaCl and concentrated on a rotary evaporator to a yellow syrup. This was distilled in a high vacuum (1.8×10⁻³ mbar) at 100° C. This gave 15.0 g of 4-(allyloxy-(2,2-dimethyl-1,3-dioxolan-4-yl)methyl)-2,2-dimethyl-1,3-dioxolane as a colorless syrup, yield 85%.

δ[¹H, CDCl₃]=1.19-1.35 (m, 12H); 3.38-3.50 (m, 2H); 3.66-3.82 (m, 2H); 3.84-4.01 (m, 4H); 4.01-4.14 (m, 1H); 5.06 (dt, 1H); 5.15 (dq, 1H); 5.76 (ddt, 1H) ppm

δ[¹³C, CDCl₃]: 25.26; 25.43; 26.15; 26.21; 26.87; 26.97; 65.60; 65.91; 70.54; 72.40; 75.53; 76.33; 78.38; 108.91; 109.47; 109.57; 116.93; 117.14; 134.28 ppm

### Example 2

### Polymer Production

### a) Homopolymerization

A solution of 740 mg of the bisacetonide prepared in example 1b) (2.7 mmol) and 9.6 mg of benzoyl peroxide (BPO, 0.04 mmol, 1.3 wt.-% based on the monomer) in 2 ml of toluene was degassed while passing nitrogen through it and then heated in a sealed flask under N₂ for 23 h at 70° C. An additional 23 mg BPO (0.09 mmol) was added and the reaction was continued at 80° C. for approximately 96 h. Subsequently, 67.2 mg BPO (0.27 mmol) were added again and the reaction was continued at 80° C. for about a further 96 h. Thereafter, the resulting polymer was precipitated by dropping in methanol/water (30:70/v:v).

60.5 mg of the polymer were dissolved in 10 ml of dichloromethane, treated with 2 ml of trifluoroacetic acid and a few drops of H₂O and stirred for 2 days at room temperature to remove the protective groups. The polymer was characterized by gel permeation chromatography (eluent DMF with 0.1% LiBr). For molecular weight calibration polystyrene standards were used.

Mn=2300 g mol⁻¹, M_{w}=7000 g mol⁻¹.

b) Copolymerization with Maleic Anhydride

A solution of 308 mg of the bisacetonide prepared in example 1b) (1.1 mmol), 115 mg of maleic anhydride (1.2 mmol) and 12 mg of azobisisobutyronitrile (AIBN, 0.07 mmol, 3.0 wt.-% based on the allyloxy monomer) in 1 ml of methanol was degassed while passing nitrogen through it and then heated in the sealed flask under N₂ for about 18 h at 65° C. Then, another 36.2 mg of AIBN (0.22 mmol, 8.6 wt %) was added and the reaction was continued at 65° C. for about 117 h. The turnover after this step is around 75%. The polymer was precipitated by dropwise addition of the reaction mixture in 25 ml of diethyl ether.

60.5 mg of the polymer were dissolved in 10 ml of dichloromethane, treated with 2 ml of trifluoroacetic acid and a few drops of H₂O and stirred for 2 days at room temperature to remove the protective groups. The polymer was characterized by gel permeation chromatography (eluent DMF with 0.1% LiBr). For molecular weight calibration polystyrene standards were used.

Mₙ=7000 g mol⁻¹, M_{w}=15000 g mol⁻¹.

c) Copolymerization with Methylmaleimide

A solution of 308 mg of the bisacetonide prepared in example 1b) (1.1 mmol), 126 mg of methylmaleimide (1.1 mmol) and 12 mg of azobisisobutyronitrile (AIBN, 0.07 mmol, 3.0 wt.-% relative on allyloxy monomer) in 1 ml of toluene was degassed while passing nitrogen through it and then heated in the sealed flask under N₂ for about 18 h at 80° C. Then, another 36 mg of AIBN (0.22 mmol, 8.3 wt %) was added and the reaction was continued at 80° C. for about 23 h. Thereafter, the polymer was precipitated by dropwise addition in 20 ml of diethyl ether. The precipitated polymer was characterized by GPC in dimethylformamide (DMF) with 0.1% LiBr. For molecular weight calibration polystyrene standards were used.

60.5 mg of the polymer were dissolved in 10 ml of dichloromethane, treated with 2 ml of trifluoroacetic acid and a few drops of H₂O and stirred for 2 days at room temperature to remove the protective groups. The polymer was characterized by gel permeation chromatography (eluent DMF with 0.1% LiBr). For molecular weight calibration polystyrene standards were used.

Mₙ=7000 gmol⁻¹, M_{w}=12000 gmol⁻¹.

### Example 3

### Washing Tests

Detergents containing active ingredients to be used according to the invention showed a significantly better primary washing performance than otherwise identically composed compositions which lacked them, or otherwise identical compositions which instead contained a polymer of the prior art.

What is claimed is:

1. A compound of general formula (I), 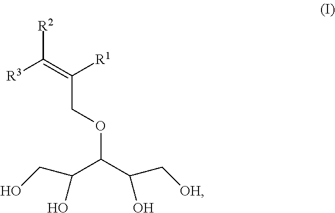
in which R¹, R² and R³ are independently H or an alkyl group having 1 to 3 carbon atoms, and their derivatives in which the hydroxyl groups are protected by protecting groups selected from acetals, ketals, and carboxylic acid esters.

2. A polymer obtainable by radical polymerization of compounds of general formula I, 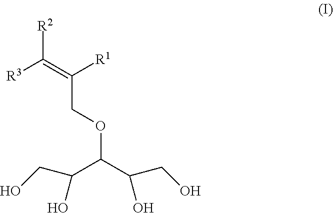
in which R¹, R² and R³ independently of one another are H or an alkyl group having 1 to 3 carbon atoms, or by their free-radical copolymerization with α,β-monoethylenically unsaturated carboxylic acids, carboxylic acid esters, carboxylic anhydrides, carboxamides, carboxylic acid imides, nitriles and their mixtures.

3. A method of removing soiling from textiles or hard surfaces by contacting the garment or surface to be cleaned with an aqueous liquor containing a detergent or cleaning product and a polymer according to claim 2.

4. A detergent or cleaning product containing a polymer according to claim 2.

5. A detergent according to claim 6, comprising from 0.1% by weight to 10% by weight of the polymer.

6. A polymer according to claim 2, wherein the polymer has an average molecular weight in the range from 1000 g/mol to 100,000 g/mol.

7. A polymer according to claim 2, obtained from the compounds of the general formula I and those derived from the α,β-monoethylenically unsaturated carboxylic acid and/or derivatives thereof, in molar ratios in the range from 4:1 to 1:4.

8. The compound, according to claim 1, wherein the hydroxyl groups are protected by acetal.

9. The compound, according to claim 1, wherein the hydroxyl groups are protected by tetrahydropyranyl ether.

10. The compound, according to claim 1, wherein the hydroxyl groups are protected by ketal.

11. The compound, according to claim 1, wherein the hydroxyl groups are protected by acetonide.

12. The compound, according to claim 1, wherein the hydroxyl groups are protected by carboxylic acid esters.

13. The compound, according to claim 1, wherein the hydroxyl groups are protected by acetate.
